(19) United States
(12) Patent Application Publication
(10) Pub. No.: US 2025/0313552 A1
(43) Pub. Date: Oct. 9, 2025

(54) NICOTINATE ESTERS AND THERAPEUTIC METHODS OF USE THEREOF

(71) Applicant: New Frontier Bio, Inc., Beverly, MA (US)

(72) Inventors: Bradley L. Pentelute, Revere, MA (US); Dinara S. Gunasekera, Cambridge, MA (US); Kevin Kong, Irvine, CA (US); Sabrina Johnson, Beverly, MA (US)

(21) Appl. No.: 19/170,296

(22) Filed: Apr. 4, 2025

Related U.S. Application Data
(60) Provisional application No. 63/574,508, filed on Apr. 4, 2024.
(60) Provisional application No. 63/664,507, filed on Jun. 26, 2024.

(51) Int. Cl. C07D 405/12; A61K 31/455; A61K 31/675; C07D 213/55; C07D 401/14; C07D 405/14; C07D 409/12; C07F 9/54

(52) U.S. Cl. CPC C07D 405/12; A61K 31/455; A61K 31/675; C07D 213/55; C07D 401/14; C07D 405/14; C07D 409/12; C07F 9/5442

(57) ABSTRACT

Disclosed are compositions and methods for increasing NAD+ in a subject (e.g., in a human subject).

### RELATED APPLICATIONS

This application claims the benefit of priority to U.S. Provisional Application Nos.: 63/664,507, filed Jun. 26, 2024; and 63/574,508, filed Apr. 4, 2024.

### BACKGROUND

Nicotinamide adenine dinucleotide (NAD) and its derivative compounds are known as essential coenzymes in cellular redox reactions in all living organisms. Several lines of evidence have also shown that NAD participates in a number of important signaling pathways in mammalian cells, including poly-ADP-ribosylation in DNA repair (Menissier de Murcia et al., EMBO J., 22:2255-2263 (2003)), mono-ADP-ribosylation in the immune response and G-protein coupled signaling (Corda and Di Girolamo, EMBO J., 22:1953-8 (2003)), and the synthesis of ADP-cyclic ribose and nicotinate adenine dinucleotide phosphate (NAADP) in intracellular calcium signaling (Lee, Annu. Rev. Pharmacol. Toxicol., 41:317-345 (2001)). Recently, NAD and its derivatives have also been shown to play an important role in transcriptional regulation (Lin and Guarente, Curr. Opin. Cell. Biol., 15:241-246 (2003)). In particular, the discovery of Sir2 NAD-dependent deacetylase activity (e.g., Imai et al., Nature, 403:795-800 (2000); Landry et al., Biochem. Biophys. Res. Commun., 278:685-690 (2000); Smith et al., Proc. Natl. Acad. Sci. USA, 97:6658-6663 (2000)) drew attention to this new role for NAD.

NAD+ is thought to be related to the aging process. This is demonstrated in the replicative life span of S. cerevisiae, which is typically defined as the number of buds or “daughter cells” produced by an individual “mother cell” (Barton, A., J. Gen. Microbiol., 4:84-86 (1950)). Mother cells undergo age-dependent changes including an increase in size, a slowing of the cell cycle, enlargement of the nucleolus, an increase in steady-state NAD+ levels, increased gluconeogenesis and energy storage, and sterility resulting from the loss of silencing at telomeres and mating-type loci (Sinclair et al., Science, 277 (5330): 1313-6 (1997); Mortimer et al., Nature, 183:1751-1752 (1959); Kennedy et al., J. Cell Biol., 127 (6): 1985-93 (1994); Kim et al., Biochem. Biophys. Res. Commun., 219 (2): 370-6 (1996); Ashrafi et al., Genes Dev., 14 (15): 1872-85 (2000); Lin et al., J. Biol. Chem., (2001)).

A key regulator of aging in yeast is the Sir2 silencing protein (Kaeberlein et al., Genes Dev., 13 (19): 2570-80 (1999)), a nicotinamide adenine dinucleotide (NAD+)-dependent deacetylase (Tanner et al., Proc. Natl. Acad. Sci. USA, 97 (26) 14178-82 (2000); Imai et al., Nature, 403 (6771): 795-800 (2000); Smith et al., Proc. Natl. Acad. Sci. USA, 97 (12): 6658-63 (2000); Landry et al., Proc. Natl. Acad. Sci. USA, 97 (11): 5807-11 (2000)). Sir2 is a component of the heterotrimeric Stir2/3/4 complex that catalyzes the formation of silent heterochromatin at telomeres and the two silent mating-type loci (Laurenson et al., Microbiol. Rev., 56 (4): 543-60 (1992)). Sir2 is also a component of the RENT complex that is required for silencing at the rDNA locus and exit from telophase (Straight et al., Cell, 97 (2): 245-56 (1999); Shou et al., Cell, 97 (2): 233-44 (1999)). This complex has also recently been shown to directly stimulate transcription of rRNA by Pol I and to be involved in regulation of nucleolar structure (Shou et al., Mol. Cell., 8 (1): 45-55 (2001)).

Biochemical studies have shown that Sir2 can readily deacetylate the amino-terminal tails of histones H3 and H4, resulting in the formation of 1-O-acetyl-ADP-ribose and nicotinamide (Tanner et al., Proc. Natl. Acad. Sci. USA, 97 (26) 14178-82 (2000); Imai et al., Nature, 403 (6771): 795-800 (2000); Smith et al., Proc. Natl. Acad. Sci. USA, 97 (12): 6658-63 (2000); Tanny et al., Proc. Natl. Acad. Sci. USA, 98 (2): 415-20 (2001)). Strains with additional copies of SIR2 display increased rDNA silencing (Smith et al., Mol. Cell Biol., 19 (4): 3184-97 (1999)) and a 30% longer life span (Kaeberlein et al., Genes Dev 13 (19): 2570-80 (1999)). It has recently been shown that additional copies of the C. elegans SIR2 homolog, sir-2.1, greatly extend life span in that organism (Tissenbaum et al., Nature, 410 (6825): 227-30 (2001)). This implies that the SIR2-dependent regulatory pathway for aging arose early in evolution and has been well conserved (Kenyon, C., Cell, 105:165-168 (2001)).

In most organisms, there are two pathways of NAD+biosynthesis. NAD+ may be synthesized de novo from tryptophan or recycled in four steps from nicotinamide via the NAD+salvage pathway. The first step in the bacterial NAD+salvage pathway, the hydrolysis of nicotinamide to nicotinic acid and ammonia, is catalyzed by the pncA gene product (Foster et al., J Bacteriol, 137 (3): 1165-75 (1979)). An S. cerevisiae gene with homology to pncA, YGL037, was recently assigned the name PNCI (SGD) (Ghislain et al., Yeast, 19 (3): 215-224 (2002)). A nicotinate phosphoribosyltransferase, encoded by the NPTI gene in S. cerevisiae, converts the nicotinic acid from this reaction to nicotinic acid mononucleotide (NaMN) (Wubbolts et al., J. Biol. Chem., 265 (29): 17665-72 (1990); Vinitsky et al., J. Bacteriol., 173 (2): 536-40 (1991); Imsande, J. Biochim. Biophys. Acta., 85, 255-273 (1964); Grubmeyer et al., Methods Enrymol., 308:28-48 (1999)). At this point, the NAD+salvage pathway and the de novo NAD+pathway converge and NaMN is converted to desamido-NAD+ (NaAD) by a nicotinate mononucleotide adenylyltransferase (NaMNAT). In S. cerevisiae, there are two putative open reading frames (ORFs) with homology to bacterial NaMNAT genes, YLR328 (Emanuelli et al., FEBS Lett., 455 (1-2): 13-7 (1999)) and an uncharacterized ORF, YGR010 (Smith et al., Proc. Natl. Acad. Sci. USA, 97 (12): 6658-63 (2000); Emanuelli et al., FEBS Lett., 455 (1-2): 13-7 (1999)). In Salmonella, the final step in the regeneration of NAD+ is catalyzed by an NAD synthetase (Hughes et al., J. Bacteriol., 170 (5): 2113-20 (1988)).

Sir2 is a limiting component of yeast longevity. A single extra copy of the SIR2 gene extends the yeast life span by 40% (Kaeberlein et al., Genes Dev., 13 (19): 2570-80 (1999); Lin et al., Science, 289 (5487): 2126-8 (2000); Anderson et al., J. Biol. Chem., 277 (21): 18881-90 (2002)). Recently, it has been shown that increased dosage of the Sir2 homologue sir2.1 extends the life span of the nematode C. elegans (Tissenbaum et al., Nature, 410 (6825): 227-30 (2001)). The nearest human homologue SIRT1, has been shown to inhibit apoptosis through deacetylation of p53 (Vaziri et al., Cell, 107 (2): 149-59 (2001); Luo et al., Cell, 107 (2): 137-48 (2001)). These findings suggest that Sir2 and its homologues have a conserved role in the regulation of survival at the cellular and organismal level.

Recently, a great deal of insight has been gained into the biochemistry of Sir2-like deacetylases (reviewed by Moazed, D., Curr Opin Cell Biol, 13 (2): 232-8 (2001)). In vitro, Sir2 has specificity for lysine 16 of histone H4 and lysines 9 and 14 of histone H3 (Imai et al., Nature, 403:795-800 (2000); Landry et al., Biochem. Biophys. Res. Commun., 278:685-690 (2000); Smith et al., Proc. Natl. Acad. Sci. USA, 97:6658-6663 (2000)). The Sir2 reaction requires NAD+ as a cofactor, allowing regulation of Sir2 activity through changes in availability of this co-substrate (Imai et al., Nature, 403:795-800 (2000); Landry et al., Biochem. Biophys. Res. Commun., 278:685-690 (2000); Smith et al., Proc. Natl. Acad. Sci. USA, 97:6658-6663 (2000); Tanner et al., Proc. Natl. Acad. Sci. USA, 97 (26) 14178-82 (2000)). Sir2 deacetylation is coupled to cleavage of the high-energy glycosidic bond that joins the ADP-ribose moiety of NAD+ to nicotinamide. Upon cleavage, Sir2 catalyzes the transfer of an acetyl group to ADP-ribose (Smith et al., Proc. Natl. Acad. Sci. USA, 97:6658-6663 (2000); Tanner et al., Proc. Natl. Acad. Sci. USA, 97 (26) 14178-82 (2000); Tanny et al., Proc. Natl. Acad. Sci. USA, 98 (2): 415-20 (2001); Sauve et al., Biochemistry, 40 (51): 15456-63 (2001)). The product of this transfer reaction is O-acetyl-ADP-ribose, a novel metabolite, which has recently been shown to cause a delay/block in the cell cycle and oocyte maturation of embryos (Borra et al., J Biol Chem, 277 (15): 12632-41 (2002)).

The other product of deacetylation is nicotinamide, a precursor of nicotinic acid and a form of vitamin B3 (Dietrich, L. S., Amer. J. Clin. Nut., 24:800-804 (1971)). High doses of nicotinamide and nicotinic acid are often used interchangeably to self-treat a range of conditions including anxiety, osteoarthritis, psychosis, and nicotinamide is currently in clinical trials as a therapy for cancer and type I diabetes (Kaanders et al., Int. J. Radiat. Oncol. Biol. Phys., 52 (3): 769-78 (2002)). Despite the important biological role of NAD+ and its association with the aging process, there still exists a need for methods of forming NAD+ precursors in a simple and cost-effective manner.

### SUMMARY OF THE INVENTION

In one aspect, the present disclosure provides compounds having a structure represented by Formula I or a pharmaceutically acceptable salt thereof:

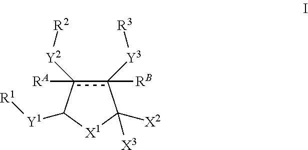

wherein
[symbol] is a single bond or a double bond;
X¹ is O, S, or N(R⁴);
X² and X³ are each independently H, alkyl, or heteroaryl; or X² and X³ combine to form ═O, ═S, or ═N(R⁴);
Y¹ is alkylenyl;
Y² is O, S, or N(R⁵);
Y³ is O, S, or N(R⁶);
R^{A} and R^{B} are each independently absent, H, or alkyl;
R¹ is H, alkyl, alkenyl, alkynyl, cycloalkyl, halo, hydroxy, carboxyl, acyl, acetyl, ester, thioester, alkyl-C(O)—O—, heteroaryl-C(O)—O—, alkoxy, phosphoryl, amino, amido, cyano, azido, or sulfonamido;
R² and R³ are each independently H, alkyl, alkenyl, alkynyl, cycloalkyl, acyl, aryl-C(O)—, heteroaryl-C(O)—, aryl, heteroaryl, aralkyl, heteroaralkyl, or heterocyclyl; and
R⁴, R⁵, and R⁶ are each independently H, alkyl, or aralkyl.

In another aspect, disclosed herein are compositions comprising a compound disclosed herein; and a pharmaceutically acceptable excipient.

In yet another aspect, disclosed herein are methods of increasing the level of NAD+ in a cell comprising contacting the cell with a compound disclosed herein, or a pharmaceutically acceptable salt thereof, under conditions effective to increase the level of NAD+ in the cell.

In yet another aspect, disclosed herein are methods of increasing intercellular NAD+ in a subject, comprising administering to a subject in need thereof a compound disclosed herein, or a pharmaceutically acceptable salt thereof, in an amount effective to increase the intercellular NAD+ in the subject.

In yet another aspect, disclosed herein are methods of treating a skin condition in a subject in need thereof, comprising administering a therapeutically effective amount of a compound, or a pharmaceutically acceptable salt thereof, to the subject.

In yet another aspect, disclosed herein are methods of treating a disease or disorder associated with cell death in a subject in need thereof, comprising administering a therapeutically effective amount of a compound, or a pharmaceutically acceptable salt thereof, to the subject.

In yet another aspect, disclosed herein are methods of treating a disease or disorder in a subject in need thereof, comprising administering a therapeutically effective amount of a compound, or a pharmaceutically acceptable salt thereof, to the subject.

### DETAILED DESCRIPTION OF THE INVENTION

In one aspect, the present disclosure provides compounds having a structure represented by Formula I or a pharmaceutically acceptable salt thereof:

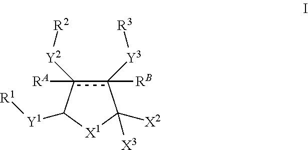

wherein
[symbol] is a single bond or a double bond;
X¹ is O, S, or N(R⁴);
X² and X³ are each independently H, alkyl, or heteroaryl; or X² and X³ combine to form ═O, ═S, or ═N(R⁴);
Y¹ is alkylenyl;
Y² is O, S, or N(R⁵);
Y³ is O, S, or N(R⁶);
R^{A} and R^{B} are each independently absent, H, or alkyl;
R¹ is H, alkyl, alkenyl, alkynyl, cycloalkyl, halo, hydroxy, carboxyl, acyl, acetyl, ester, thioester, alkyl-C(O)—O—, heteroaryl-C(O)—O—, alkoxy, phosphoryl, amino, amido, cyano, azido, or sulfonamido;
R² and R³ are each independently H, alkyl, alkenyl, alkynyl, cycloalkyl, acyl, aryl-C(O)—, heteroaryl-C(O)—, aryl, heteroaryl, aralkyl, heteroaralkyl, or heterocyclyl; and
R⁴, R⁵, and R⁶ are each independently H, alkyl, or aralkyl.

In certain embodiments, [symbol] is a single bond. In certain embodiments, R^{A} is H. In certain embodiments, R^{B} is H. In certain preferred embodiments, [symbol] is a double bond; and R^{A} and R^{B} are each absent.

In certain embodiments, X¹ is O.

In certain embodiments, X² is heteroaryl (e.g., pyridinyl or a salt thereof, such as amino- or amido-substituted pyridinyl or a salt thereof).

In certain embodiments, X³ is H.

In certain preferred embodiments, X² and X³ combine to form=O.

In certain embodiments, Y¹ is C₁-C₁₀alkylenyl. In certain preferred embodiments, Y¹ is C₂alkylenyl.

In certain embodiments, Y¹ is substituted with alkyl, alkenyl, alkynyl, halo, hydroxy, oxo, carboxyl, acyl, acetyl, ester, thioester, alkoxy, phosphoryl, amino, amido, cyano, nitro, azido, alkylthio, cycloalkyl, alkylsulfonyl, and sulfonamido. In certain preferred embodiments, Y¹ is substituted with hydroxy. In other preferred embodiments, Y¹ is substituted with ester (e.g., heteroaryl ester, such as pyridyl ester).

In certain embodiments, R¹ is alkyl-C(O)—O— (e.g., C₁₅alkyl-C(O)—O—) or heteroaryl-C(O)—O— (e.g., pyridyl-C(O)—O—). In certain embodiments, R¹ is alkyl-C(O)—O— substituted with heterocyclyl (e.g., dithiolane).

In certain embodiments, Y² is O.

In certain embodiments, R² is H. In other embodiments, R² is alkyl-C(O)— (e.g., C₁₅alkyl-C(O)—) or heteroaryl-C(O)— (e.g., pyridyl-C(O)—). In certain embodiments, R² is alkyl-C(O)— substituted with heterocyclyl (e.g., dithiolane).

In certain embodiments, Y³ is O.

In certain embodiments, R³ is H. In other embodiments, R³ is alkyl-C(O)— (e.g., C₁₅alkyl-C(O)—) or heteroaryl-C(O)— (e.g., pyridyl-C(O)—). In yet other embodiments, R³ is heterocyclyl (e.g., pyranyl, such as tetrahydropyranyl). In certain embodiments, R³ is alkyl-C(O)— substituted with heterocyclyl (e.g., dithiolane).

In certain embodiments, R³ is substituted with alkyl, alkenyl, alkynyl, halo, hydroxy, oxo, carboxyl, acyl, acetyl, ester, thioester, alkoxy, phosphoryl, amino, amido, cyano, nitro, azido, alkylthio, cycloalkyl, alkylsulfonyl, and sulfonamido.

In certain embodiments, R³ is substituted with ester (e.g., heteroarylester, such as pyridylester).

In certain embodiments, the compound is selected from the group consisting of

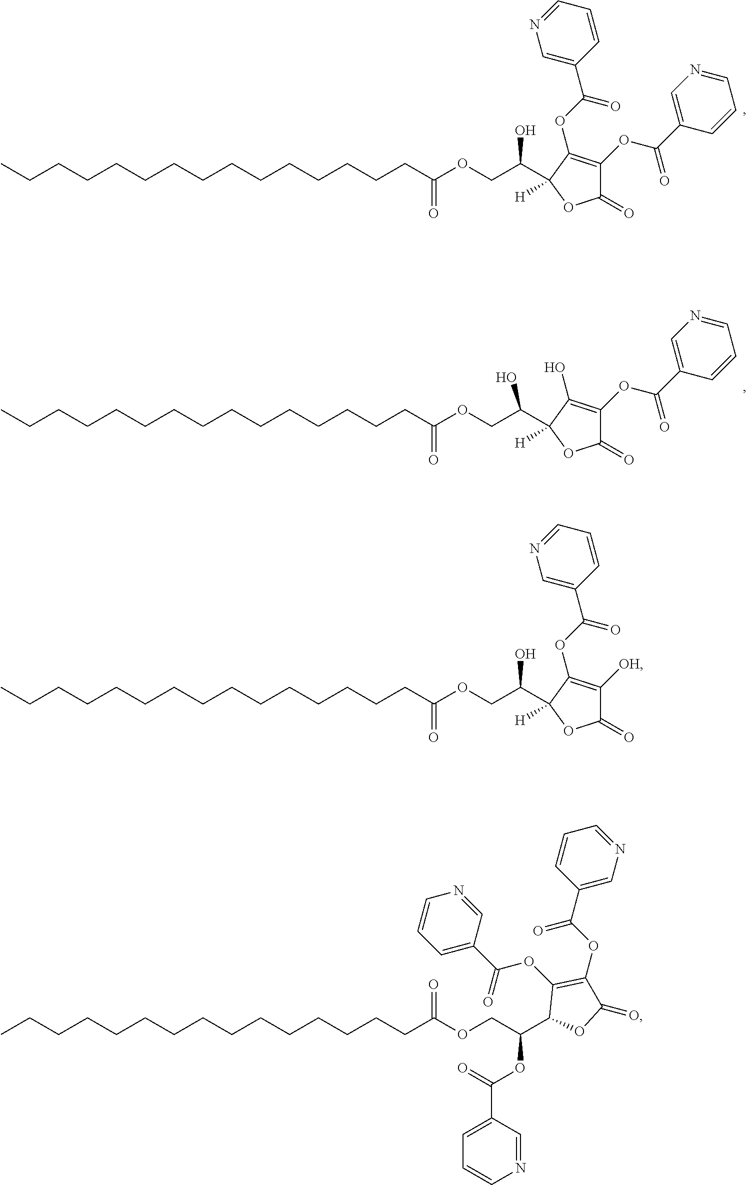 [SMILES: CCCCCCCCCCCCCCCC(=O)OC[C@@H](O)[C@@H]1OC(=O)C(OC(=O)c2cccnc2)=C1OC(=O)c1cccnc1 ; CCCCCCCCCCCCCCCC(=O)OC[C@@H](O)[C@@H]1OC(=O)C(OC(=O)c2cccnc2)=C1O ; CCCCCCCCCCCCCCCC(=O)OC[C@@H](O)[C@@H]1OC(=O)C(O)=C1OC(=O)c1cccnc1 ; CCCCCCCCCCCCCCCC(=O)OC[C@H](OC(=O)c1cccnc1)[C@H]1OC(=O)C(OC(=O)c2cccnc2)=C1OC(=O)c1cccnc1]

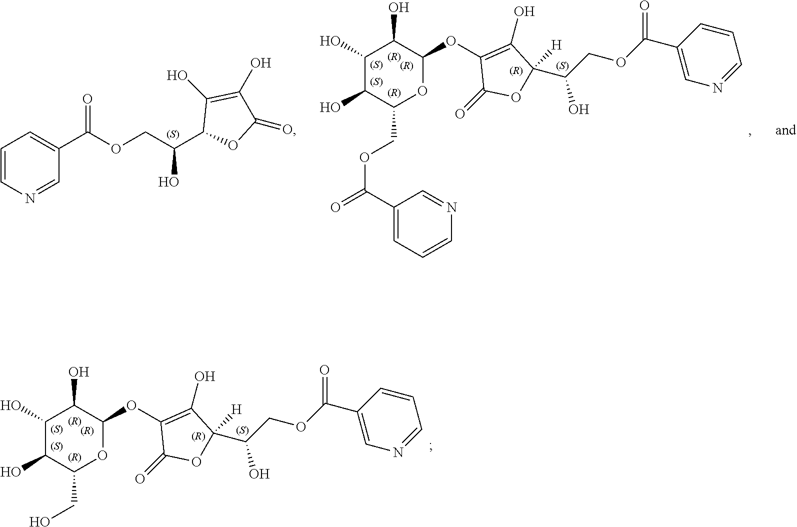

or a pharmaceutically acceptable salt thereof.

In certain embodiments, the compound is selected from the group consisting of

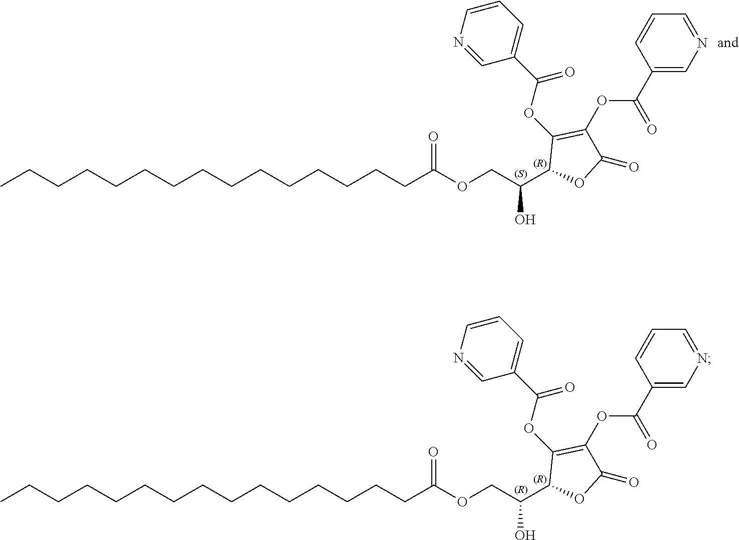

; or a pharmaceutically acceptable salt thereof.

In another aspect, the present disclosure provides a compound selected from the group consisting of:

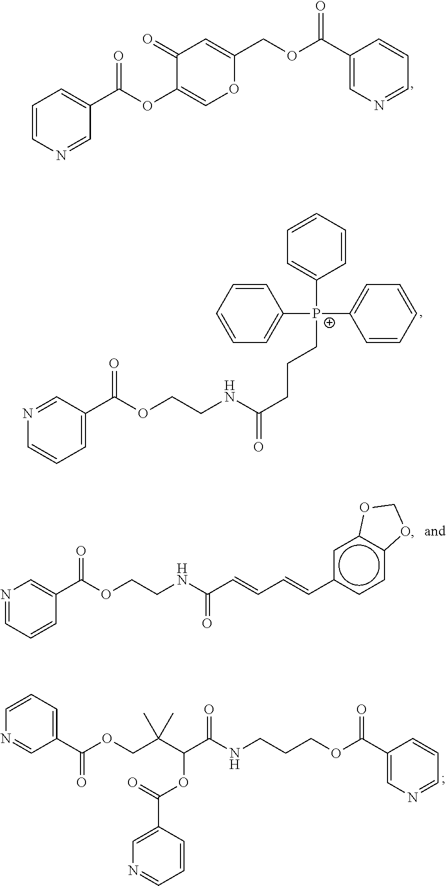

or a pharmaceutically acceptable salt thereof.

In another aspect, the present disclosure provides a compound, wherein the compound is

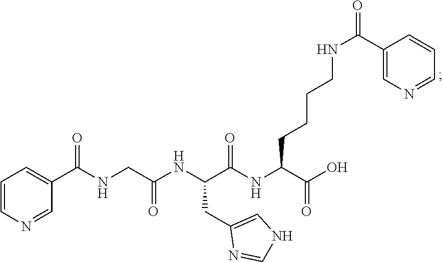 [SMILES: O=C(CNC(=O)c1cccnc1)N[C@@H](Cc1c[nH]cn1)C(=O)N[C@@H](CCCCNC(=O)c1cccnc1)C(=O)O]

or a pharmaceutically acceptable salt thereof.

In another aspect, disclosed herein are compositions comprising a compound disclosed herein and a pharmaceutically acceptable excipient. In certain preferred embodiments, the composition is formulated to topical administration.

In yet another aspect, disclosed herein are methods of increasing the level of NAD+ in a cell comprising contacting the cell with a compound disclosed herein, or a pharmaceutically acceptable salt thereof, under conditions effective to increase the level of NAD+ in the cell. In certain embodiments, the subject is a human.

In yet another aspect, disclosed herein are methods of treating a skin condition in a subject in need thereof, comprising administering a therapeutically effective amount of a compound, or a pharmaceutically acceptable salt thereof, to the subject. In certain embodiments, the skin condition is associated with or caused by inflammation, sun damage, or aging. In certain embodiments, the skin condition is selected from the group consisting of contact dermatitis, irritant contact dermatitis, allergic contact dermatitis, atopic dermatitis, actinic keratosis, keratinization disorders, eczema, epidermolysis bullosa diseases, exfoliative dermatitis, seborrheic dermatitis, erythema multiformed, erythema nodosum, damage caused by the sun or other light sources, discoid lupus crythematosus, dermatomyositis, psoriasis, skin cancer, and the effects of aging. In certain embodiments, the composition is administered topically, to the skin as an ointment, lotion, cream, microemulsion, gel, or solution.

In yet another aspect, disclosed herein are methods of treating a disease or disorder associated with cell death in a subject in need thereof, comprising administering a therapeutically effective amount of a compound, or a pharmaceutically acceptable salt thereof, to the subject. In certain embodiments, the disease or disorder is associated with neural cell death, neuronal dysfunction, or muscular cell death or dysfunction. In certain embodiments, the disease or disorder is selected from the group consisting of Parkinson's disease; Alzheimer's disease; multiple sclerosis; amyotropic lateral sclerosis; muscular dystrophy; AIDS; fulminant hepatitis; Creutzfeld-Jakob disease; retinitis pigmentosa; cerebellar degeneration; myelodysplasis; aplastic anemia; ischemic diseases; myocardial infarction; stroke; hepatic diseases; alcoholic hepatitis; hepatitis B; hepatitis C; osteoarthritis; atherosclerosis; alopecia; damage to the skin due to UV light; lichen planus; atrophy of the skin; cataract; graft rejections; and cell death caused by surgery, drug therapy, chemical exposure or radiation exposure.

In yet another aspect, disclosed herein are methods of treating a disease or disorder in a subject in need thereof, comprising administering a therapeutically effective amount of a compound, or a pharmaceutically acceptable salt thereof, to the subject. In certain embodiments, the disease or disorder is selected from the group consisting of Parkinson's disease; Alzheimer's disease; multiple sclerosis; amyotropic lateral sclerosis; muscular dystrophy; AIDS; fulminant hepatitis; Creutzfeld-Jakob disease; retinitis pigmentosa; cerebellar degeneration; myelodysplasis; aplastic anemia; ischemic diseases; myocardial infarction; stroke; hepatic diseases; alcoholic hepatitis; hepatitis B; hepatitis C; osteoarthritis; atherosclerosis; alopecia; damage to the skin due to UV light; lichen planus; atrophy of the skin; cataract; graft rejections; and cell death caused by surgery, drug therapy, chemical exposure or radiation exposure.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, dicthanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, l-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC (O)—, preferably alkylC (O)—.

The term “acylamino” is art-recognized and refers to an amino group substituted with an acyl group and may be represented, for example, by the formula hydrocarbylC (O) NH—.

The term “acyloxy” is art-recognized and refers to a group represented by the general formula hydrocarbylC (O)O—, preferably alkylC (O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc. The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁-6alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “amido”, as used herein, refers to a group

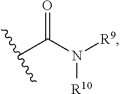

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

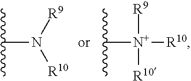

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

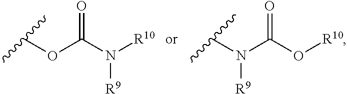

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a-O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl(in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamido” is art-recognized and refers to the group represented by the general formulae

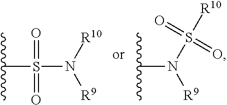

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group. The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

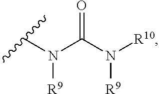

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention and are not intended to limit the invention.

### Example 1: Synthesis of Exemplary Compounds of the Disclosure

### Compound 1: (S)-2-((R)-1-hydroxy-2-(palmitoyloxy)ethyl)-5-oxo-2,5-dihydrofuran-3,4-diyl dinicotinate

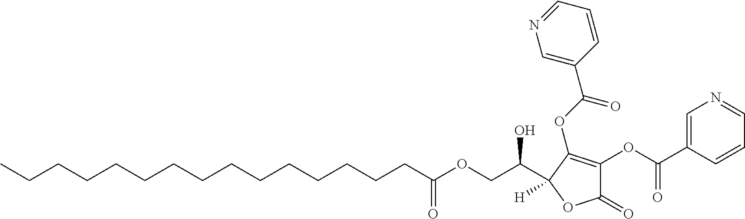 [SMILES: CCCCCCCCCCCCCCCC(=O)OC[C@@H](O)[C@@H]1OC(=O)C(OC(=O)c2cccnc2)=C1OC(=O)c1cccnc1]

To a solution of ascorbyl palmitate (1 g, 2.41 mmol, 1.00 eq) and nicotinoyl chloride (0.95 g, 5.34 mmol, 2.20 eq) in acetonitrile (4 mL) was added N,N-Diisopropylethylamine (2.0 mL, 1.56 g, 12.09 mmol, 5 eq) at 25° C. The mixture was stirred at 25° C. overnight. LC-MS (RT=3.728 min) showed ascorbyl palmitate was consumed completely and desired mass was detected. The reaction mixture was concentrated under pressure to remove acetonitrile. The resulting crude, was recrystallized from methanol and water to obtain(S)-2-((R)-1-hydroxy-2-(palmitoyloxy)ethyl)-5-oxo-2,5-dihydrofuran-3,4-diyl dinicotinate (1.21 g, 81%) as a pale yellow solid. LCMS: RT=3.674 min, MS (ESI) m/z=625 [M+H]+. 1H NMR (400 MHZ, CDCl3) δ 9.49-9.39 (m, 1H), 8.95 (d, J=1.1 Hz, 1H), 8.57 (dd, J=4.9, 1.7 Hz, 1H), 8.48 (dd, J=5.0, 1.5 Hz, 1H), 8.33-8.22 (m, 1H), 7.65 (dt, J=8.0, 1.8 Hz, 1H), 7.21 (ddd, J=8.1, 5.0, 0.7 Hz, 1H), 7.08 (dd, J=7.9, 5.1 Hz, 1H), 6.04-5.89 (m, 1H), 4.76-4.49 (m, 2H), 2.44-2.24 (m, 2H), 1.70-1.51 (m, 2H), 1.43-1.15 (m, 27H), 0.87 (t, J=6.9 Hz, 3H).

### Compound 2: (S)-4-hydroxy-5-((R)-1-hydroxy-2-(palmitoyloxy)ethyl)-2-oxo-2,5-dihydrofuran-3-yl nicotinate

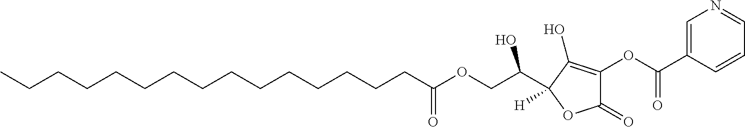 [SMILES: CCCCCCCCCCCCCCCC(=O)OC[C@@H](O)[C@@H]1OC(=O)C(OC(=O)c2cccnc2)=C1O]

To a solution of ascorbyl palmitate (1 g, 2.41 mmol, 1.00 eq) and nicotinoyl chloride (0.52 g, 2.92 mmol, 1.20 eq) in acetonitrile (2 mL) was added N,N-Diisopropylethylamine (2.0 mL, 1.56 g, 12.09 mmol, 5 eq) at 25° C. The mixture was stirred at 25° C. overnight. LC-MS showed ascorbyl palmitate was consumed completely and desired mass was detected. The reaction mixture was concentrated under pressure to remove acetonitrile. The resulting crude, was purified by prep-HPLC (column: Phenomenex luna C_{18 150×40} mm×15 um; mobile phase: [water (0.225% FA)-ACN]; B %: 80%-100%,15 min) to afford(S)-4-hydroxy-5-((R)-1-hydroxy-2-(palmitoyloxy)ethyl)-2-oxo-2,5-dihydrofuran-3-yl nicotinate as a pale yellow solid. LCMS: RT=3.562 min, MS (ESI) m/z=520 [M+H]⁺.

### Compound 3: (S)-4-hydroxy-2-((R)-1-hydroxy-2-(palmitoyloxy)ethyl)-5-oxo-2,5-dihydrofuran-3-yl nicotinate

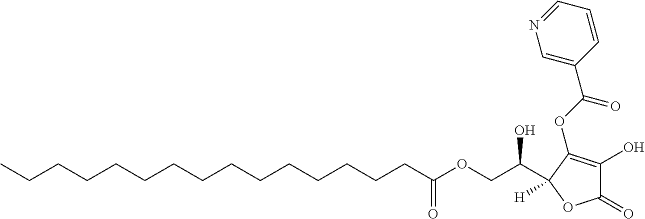 [SMILES: CCCCCCCCCCCCCCCC(=O)OC[C@@H](O)[C@@H]1OC(=O)C(O)=C1OC(=O)c1cccnc1]

To a solution of ascorbyl palmitate (1 g, 2.41 mmol, 1.00 eq) and nicotinoyl chloride (0.52 g, 2.92 mmol, 1.20 eq) in dimethylformamide (2 mL) was added N,N-Diisopropylethylamine (2.0 mL, 1.56 g, 12.09 mmol, 5 eq) at 25° C. The mixture was stirred at 25° C. overnight. LC-MS showed ascorbyl palmitate was consumed completely and desired mass was detected. The reaction mixture was concentrated under pressure to remove acetonitrile. The resulting crude, was The residue was purified by prep-HPLC (column: Phenomenex luna C18 150×40 mm×15 um; mobile phase: [water (0.225% FA)-ACN]; B %: 80%-100%,15 min) to afford(S)-4-hydroxy-2-((R)-1-hydroxy-2-(palmitoyloxy)ethyl)-5-oxo-2,5-dihydrofuran-3-yl nicotinate as a pale yellow solid. LCMS: RT=3.66 min, MS (ESI) m/z=520 [M+H]⁺.

### Compound 4: (R)-2-((S)-1-(nicotinoyloxy)-2-(palmitoyloxy)ethyl)-5-oxo-2,5-dihydrofuran-3,4-diyl dinicotinate

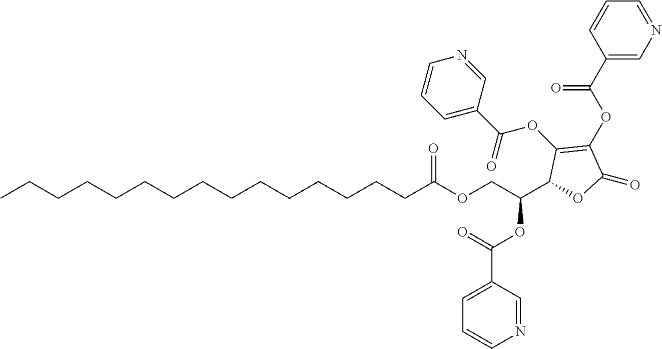 [SMILES: CCCCCCCCCCCCCCCC(=O)OC[C@H](OC(=O)c1cccnc1)[C@H]1OC(=O)C(OC(=O)c2cccnc2)=C1OC(=O)c1cccnc1]

To a solution of ascorbyl palmitate (1 g, 2.41 mmol, 1.00 eq) and nicotinoyl chloride (1.93 g, 10.84 mmol, 4.5 eq) in dimethylformamide (2 mL) was added N,N-Diisopropylethylamine (2.0 mL, 1.56 g, 12.09 mmol, 5 eq) at 25° C. The mixture was stirred at 25° C. overnight. LC-MS showed ascorbyl palmitate was consumed completely and desired mass was detected. The reaction mixture was concentrated under pressure to remove acetonitrile. The resulting crude, was The residue was purified by prep-HPLC (column: Phenomenex luna C18 150×40 mm×15 um; mobile phase: [water (0.225% FA)-ACN]; B %: 80%-100%, 15 min) to afford (R)-2-((S)-1-(nicotinoyloxy)-2-(palmitoyloxy)ethyl)-5-oxo-2,5-dihydrofuran-3,4-diyl dinicotinate as a pale yellow solid. LCMS: RT=4.465 min, MS (ESI) m/z=730 [M+H]⁺.

Compound 5: (5-(nicotinoyloxy)-4-oxo-4H-pyran-2-yl)methyl nicotinate

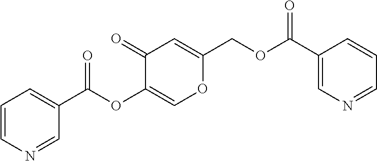 [SMILES: O=C(OCc1cc(=O)c(OC(=O)c2cccnc2)co1)c1cccnc1]

2.5 eq of Nicotinoyl chloride ((3.13 g, 17.58 mmol, 2.50 eq) hydrochloride was dissolved in 0.1 M acetonitrile and treated with 1 eq of Kojic acid (1 g, 7.04 mmol, 1.00 eq) at 0° C. The reaction was stirred for 24 hours. The crude product was purified by reversed-phase HPLC (MeCN/H2O, 0.1% TFA) to afford (5-(nicotinoyloxy)-4-oxo-4H-pyran-2-yl)methyl nicotinate in 81% yield) as white pale brown solid. LCMS: RT=2.986 min, MS (ESI) m/z=353 [M+H]⁺.

### Compound 6: (S)-2-((R)-3,4-dihydroxy-5-oxo-2,5-dihydrofuran-2-yl)-2-hydroxyethyl nicotinate

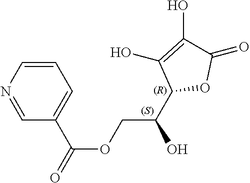

To a solution of (R)-5-((S)-1,2-dihydroxyethyl)-3,4-dihydroxyfuran-2 (5H)-one (1, 100 mg, 567.78 μmol, 105.26 μL, 1 eq) in DMF (1 mL) were added DCC (117.15 mg, 567.78 μmol, 114.85 μL, 1 eq), DMAP (34.68 mg, 283.89 μmol, 0.5 eq) and nicotinic acid (1A, 69.90 mg, 567.78 μmol, 47.45 μL, 1 eq). The mixture was stirred at 25° C. for 12 hr. LCMS (5-95AB/1.5 min, RT=0.356 min, 282.0 [M+H]+, ESI pos) showed the major peak with desired product. The reaction mixture was filtered and concentrated under reduced pressure to give a residue. The residue was purified by reversed-phase HPLC column: Phenomenex luna C18 150×25mm×10 um; mobile phase: [water (HCl)-ACN]; gradient: 1%-15% B over 10 min to afford(S)-2-((R)-3,4-dihydroxy-5-oxo-2,5-dihydrofuran-2-yl)-2-hydroxyethyl nicotinate (N-61, 33.1 mg, 115.15 μmol, 20.28% yield, 97.83% purity) as a white solid. LCMS: Rt=0.532 min, m/z=282.1 (M+H)+. 1H NMR (400 MHZ, CD₃OD) δ 9.50-9.45 (m, 1H), 9.18 (td, J=1.7, 8.2 Hz, 1H), 9.13-9.07 (m, 1H), 8.26 (dd, J=6.1, 7.9 Hz, 1H), 4.92 (br s, 1H), 4.67-4.59 (m, 2H), 4.33 (br d, J=2.1 Hz, 1H).

### Compound 7: (S)-2-hydroxy-2-((R)-3-hydroxy-5-oxo-4-(((2R,3R,4S,5S,6R)-3,4,5-trihydroxy-6-((nicotinoyloxy)methyl)tetrahydro-2H-pyran-2-yl)oxy)-2,5-dihydrofuran-2-yl)ethyl nicotinate

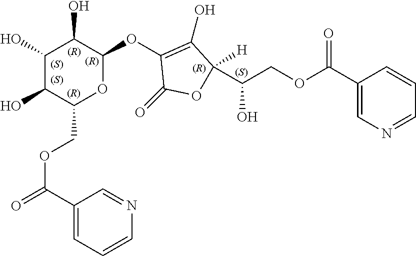

To a solution of (R)-5-((S)-1,2-dihydroxyethyl)-4-hydroxy-3-(((2S,3S,4R,5R,6S)-3,4,5-trihydroxy-6-(hydroxymethyl)tetrahydro-2H-pyran-2-yl)oxy) furan-2 (5H)-one (400.00 mg, 1.18 mmol, 1 eq) in DMF (4 mL) were added DCC (487.97 mg, 2.37 mmol, 478.41 μL, 2 eq), DMAP (288.93 mg, 2.37 mmol, 2 eq) and nicotinic acid (436.73 mg, 3.55 mmol, 296.49 μL, 3 eq). The mixture was stirred at 25° C. for 12 hr. LCMS (5-95AB/1.5 min, RT-0.401 min, 549.2 [M+H]⁺, ESI pos) showed the major peak with desired product. The reaction mixture was filtered and concentrated under reduced pressure to give a residue. The residue was purified by reversed-phase HPLC column: Phenomenex luna C_{18 150×40} mm×15 um; mobile phase: [water (TFA)-ACN];gradient: 0%-23% B over 15 min to afford(S)-2-hydroxy-2-((R)-3-hydroxy-5-oxo-4-(((2R,3R,4S,5S,6R)-3,4,5-trihydroxy-6-((nicotinoyloxy)methyl)tetrahydro-2H-pyran-2-yl)oxy)-2,5-dihydrofuran-2-yl)ethyl nicotinate (150 mg, 259.50 μmol, 21.95% yield, 94.884% purity) as a white solid. LCMS: Rt=0.393 min, m/z=549.1 (M+H)+. 1H NMR (400 MHZ, CD₃OD) δ 9.17 (d, J=1.5 Hz, 1H), 9.11 (d, J=1.6 Hz, 1H), 8.83 (ddd, J=1.6, 3.0, 4.8 Hz, 2H), 8.56 (td, J=1.8, 8.1 Hz, 1H), 8.46 (td, J=1.8, 8.0 Hz, 1H), 7.75-7.66 (m, 2H), 6.02-5.94 (m, 1H), 5.37 (d, J=2.3 Hz, 1H), 5.26 (d, J=3.6 Hz, 1H), 4.97-4.94 (m, 1H), 4.59-4.50 (m, 1H), 4.03 (td, J=3.2, 10.1 Hz, 1H), 3.75-3.65 (m, 2H), 3.62-3.58 (m, 1H), 3.50-3.40 (m, 2H).

### Compound 8: (S)-2-hydroxy-2-((R)-3-hydroxy-5-oxo-4-(((2R,3R,4S,5S,6R)-3,4,5-trihydroxy-6-(hydroxymethyl)tetrahydro-2H-pyran-2-yl)oxy)-2,5-dihydrofuran-2-yl)ethyl nicotinate

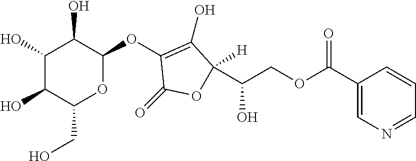 [SMILES: O=C1O[C@H]([C@@H](O)COC(=O)c2cccnc2)C(O)=C1O[C@H]1O[C@H](CO)[C@@H](O)[C@H](O)[C@H]1O]

To a solution of (R)-5-((S)-1,2-dihydroxyethyl)-4-hydroxy-3-(((2S,3S,4R,5R,6S)-3,4,5-trihydroxy-6-(hydroxymethyl)tetrahydro-2H-pyran-2-yl)oxy) furan-2 (5H)-one (1, 200 mg, 591.25 μmol, 1 eq) in DMF (2 mL) were added DCC (121.99 mg, 591.25 μmol, 119.60 μL, 1 eq), DMAP (36.12 mg, 295.63 μmol, 0.5 eq) and nicotinic acid (1A, 72.79 mg, 591.25 μmol, 49.42 μL, 1 eq). The mixture was stirred at 25° C. for 12 hours. LCMS (0-60AB/1.5 min, RT=0.345 min, 444.1 [M+H]+, ESI pos) showed the major peak with desired MS. The reaction mixture was filtered and concentrated under reduced pressure to give a residue. The crude product was purified by reversed-phase HPLC (column: Waters Atlantis T3 150×30 mm×5 um; mobile phase: [water (FA)-ACN];gradient: 1%-30% B over 10 min) to afford(S)-2-hydroxy-2-((R)-3-hydroxy-5-oxo-4-(((2R,3R,4S,5S,6R)-3,4,5-trihydroxy-6-(hydroxymethyl)tetrahydro-2H-pyran-2-yl)oxy)-2,5-dihydrofuran-2-yl)ethyl nicotinate (117.9 mg, 257.24 μmol, 43.51% yield, 96.652% purity) as a white solid. LCMS: Rt=0.349 min, m/z=444.1 (M+H)+1H NMR (400 MHZ, MeOD) δ 9.18 (d, J=1.4 Hz, 1H), 8.76 (dd, J=1.6, 4.9 Hz, 1H), 8.47 (td, J=1.9, 8.0 Hz, 1H), 7.59 (dd, J=4.9, 7.9 Hz, 1H), 5.40 (d, J=3.6 Hz, 1H), 4.95 (d, J=1.9 Hz, 1H), 4.58-4.48 (m, 2H), 4.32 (br d, J=1.8 Hz, 1H), 4.09-4.00 (m, 1H), 3.84-3.76 (m, 2H), 3.74-3.68 (m, 1H), 3.53 (dd, J=3.6, 9.8 Hz, 1H), 3.41 (dd, J=9.3, 9.9 Hz, 1H).

### Compound 9: (4-((2-(nicotinoyloxy)ethyl)amino)-4-oxobutyl)triphenylphosphonium

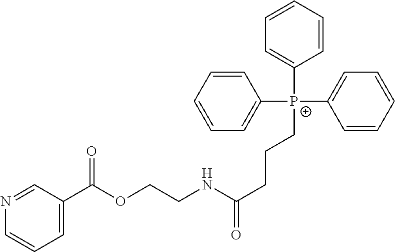 [SMILES: O=C(CCC[P+](c1ccccc1)(c1ccccc1)c1ccccc1)NCCOC(=O)c1cccnc1]

To a solution of 2-aminoethyl nicotinate (1 eq), (3-carboxypropyl)triphenylphosphonium (1 eq) in DCM (10 mL) wwas added EDCI (1.5 eq), DMAP (0.2 eq), TEA (1 eq). The mixture was stirred at 25° C. for 16 hours. LCMS showed the major peak with desired product was detected. The reaction mixture was filtered and concentrated under reduced pressure to give a crude residue. Then the residue was purified by prep-HPLC (column: Phenomenex luna C18 250×50 mm×10 um; mobile phase: [water (HCl)-ACN]; B %: 0%-25%,13 min) to (4-((2-(nicotinoyloxy)ethyl)amino)-4-oxobutyl)triphenylphosphonium (82% yield, 98% purity) as white solid. LCMS: tp=3.1 min, m/z=498.11 (M+1)⁺.

### Compound 10:2-((2E,4E)-5-(benzo[d][1,3]dioxol-5-yl) penta-2,4-dienamido)ethyl nicotinate

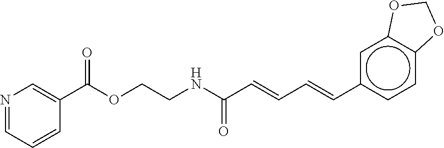

To a solution of 2-aminoethyl nicotinate (1 eq), ((2E,4E)-5-(benzo[d][1,3]dioxol-5-yl) penta-2,4-dienoic acid (1 eq) in DCM (10 mL) were added EDCI (1.5 eq), DMAP (0.2 eq), TEA (1 eq). The mixture was stirred at 25° C. for 8 hours. LCMS showed the major peak with desired product was detected. The reaction mixture was filtered and concentrated under reduced pressure to give a crude residue. Then the residue was purified by prep-HPLC (column: Phenomenex luna C18 250×50 mm×10 um; mobile phase: [water (HCl)-ACN]; B %: 0%-25%,13 min) to 2-((2E,4E)-5-(benzo[d][1,3]dioxol-5-yl) penta-2,4-dienamido)ethyl nicotinate (92% yield, 99% purity) as white solid. LCMS: tp=5.1 min, m/z=366.12 (M+1)+.

### Compound 11:2,2-dimethyl-4-((3-(nicotinoyloxy) propyl)amino)-4-oxobutane-1,3-diyl dinicotinate

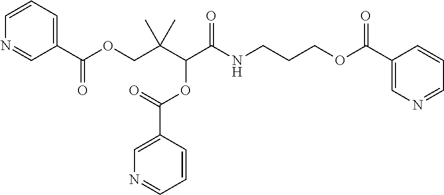 [SMILES: CC(C)(COC(=O)c1cccnc1)C(OC(=O)c1cccnc1)C(=O)NCCCOC(=O)c1cccnc1]

To a solution of Panthenol (1 eq), nicotinoyl chloride (4 eq) in DCM (10 mL) were added TEA (1 eq). The mixture was stirred at 25° C. for 8 hours. LCMS showed the major peak with desired product was detected. The reaction mixture was filtered and concentrated under reduced pressure to give a crude residue. Then the residue was purified by prep-HPLC (column: Phenomenex luna C18 250×50 mm×10 um; mobile phase: [water (HCl)-ACN]; B %: 0%-25%,13 min) to 2,2-dimethyl-4-((3-(nicotinoyloxy) propyl)amino)-4-oxobutane-1,3-diyl dinicotinate (96% yield, 99% purity) as white solid. LCMS: tp=7.7 min, m/z=521.78 (M+1)+.

### Compound 12:1-((2R,3R,4R,5R)-3,4-bis((5-(1,2-dithiolan-3-yl) pentanoyl)oxy)-5-(((5-(1,2-dithiolan-3-yl) pentanoyl)oxy)methyl)tetrahydrofuran-2-yl)-3-carbamoylpyridin-1-ium (NFB 1124)

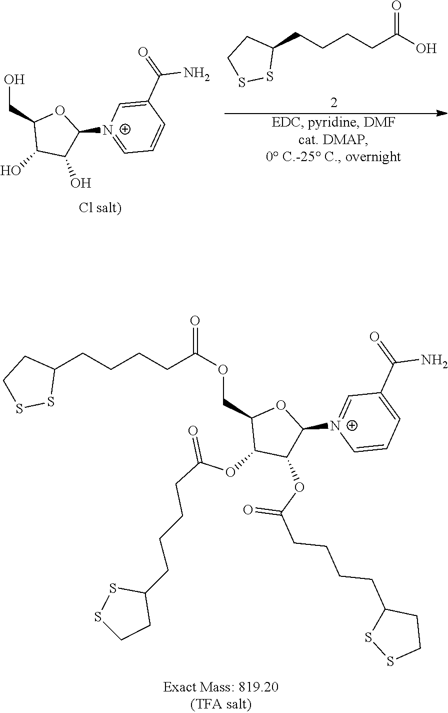 [SMILES: NC(=O)c1ccc[n+]([C@@H]2O[C@H](CO)[C@@H](O)[C@H]2O)c1 ; O=C(O)CCCC[C@@H]1CCSS1 ; NC(=O)c1ccc[n+]([C@@H]2O[C@H](COC(=O)CCCCC3CCSS3)[C@@H](OC(=O)CCCCC3CCSS3)[C@H]2OC(=O)CCCCC2CCSS2)c1]

Nicotinamide riboside chloride 1 (200 mg, 0.690 mmol, 1.00 equiv.) and a-lipoic acid 2 (805 mg, 3.91 mmol, 4.99 equiv.) were suspended in acetonitrile (6 mL) and cooled to 0° C. with stirring before adding EDC (750 mg, 3.91 mmol, 4.99 equiv.). Pyridine (310 μL, 304 mg, 3.90 mmol, 4.97 equiv.) was added dropwise at 0° C., followed by catalytic DMAP (10 mg, 0.08 mmol, 10 mol %). The reaction was allowed to warm to 25° C. and stir overnight. LC-MS (DG1-72-4, RT=8.114 min) indicated that Nicotinamide riboside chloride was consumed and the desired mass (m/z 819.3) was detected. The acetonitrile was removed under pressure, and the resulting residue was purified directly using C₁₈ reverse-phase chromatography to obtain 3 (514 mg, 91%) as a yellow solid. LCMS: Product: RT=8.704 min, MS (ESI) m/z=819.3 [M+H]⁺.

### Compound 13:1-((2R,3R,4R,5R)-3,4-bis(nicotinoyloxy)-5 ((nicotinoyloxy)methyl)tetrahydrofuran-2-yl)-3-carbamoylpyridin-1-ium

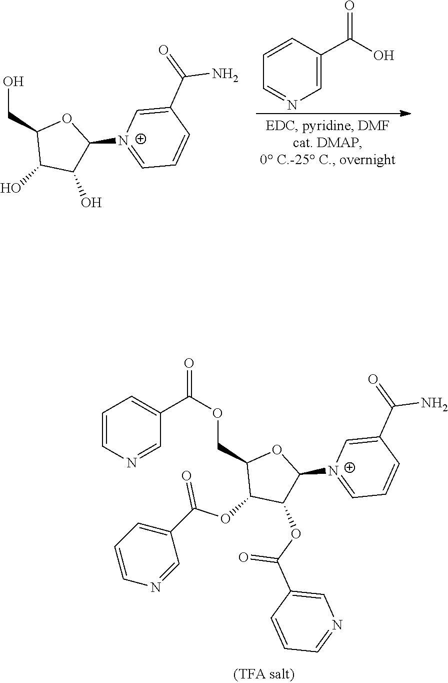 [SMILES: NC(=O)c1ccc[n+]([C@@H]2O[C@H](CO)[C@@H](O)[C@H]2O)c1 ; O=C(O)c1cccnc1 ; NC(=O)c1ccc[n+]([C@@H]2O[C@H](COC(=O)c3cccnc3)[C@@H](OC(=O)c3cccnc3)[C@H]2OC(=O)c2cccnc2)c1]

Nicotinamide riboside chloride (300 mg, 1.18 mmol, 1.00 equiv.) and nicotinic acid 5 (724 mg, 5.89 mmol, 4.99 equiv.) were suspended in acetonitrile (9 mL) and cooled to 0° C. with stirring before adding EDC (1.120 g, 5.84 mmol, 4.95 equiv.). Pyridine (465 μL, 457 mg, 5.86 mmol, 4.97 equiv.) was added dropwise at 0° C., followed by catalytic DMAP (10 mg, 0.08 mmol, 7 mol %). The reaction was allowed to warm to 25° C. and stir overnight. LC-MS (RT=8.114 min) indicated that Nicotinamide riboside chloride was consumed, and the desired mass (m/z 570.2) was detected. The acetonitrile was removed under pressure, and the resulting residue was purified directly using C18 reverse-phase chromatography to obtain 3 (585 mg, 91%) as a yellow solid. LCMS: roduct: RT=8.704 min, MS (ESI) m/z=570.2 [M+H]⁺.

### Compound 14: (R)-2-((S)-1-hydroxy-2-(palmitoyloxy)ethyl)-5-oxo-2,5-dihydrofuran-3,4-diyl dinicotinate

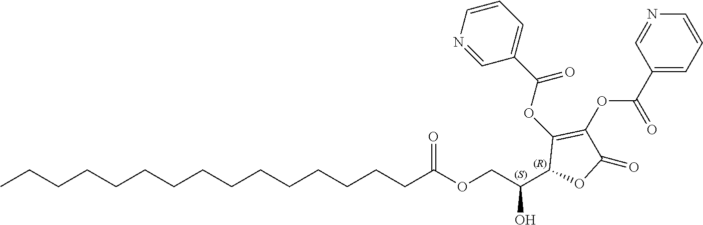

To a solution of 6-O-Palmitoyl-L-ascorbic acid (170 g, 410 mmol, 1 eq) and nicotinoyl chloride (183 g, 1.03 mol, 2.5 eq) in acetonitrile (2.50 L) was added N,N-Diisopropylethylamine (265 g, 2.05 mol, 357 mL, 5 eq) at 25° C. The mixture was stirred at 25° C. for 16 hrs. HPLC (EW54182-3-P1A1) showed the 6-O-Palmitoyl-L-ascorbic acid was consumed completely, and the peak of product was found. Two batches of reaction mixture were combined and concentrated to remove acetonitrile under reduced pressure. Then diluted with EtOAc (1.50 L), and H₂O (1.50 L), separated, extracted with EtOAc (1.00 L). The combined organic layers were washed with H₂O (1.50 L*3), brine (500 ml), dried over Na₂SO₄, filtered and concentrated under reduced pressure to get the residue. The crude product was purified by reversed-phase HPLC (0.1% NH₃·H₂O) and was concentrated under reduced pressure to remove the most acetonitrile. Extracted with EtOAc (8.00 L*2). The combined organic layers were washed with brine (5.00 L), dried over Na₂SO₄, filtered and concentrated under reduced pressure to get the residue. The residue was resolved with CH₂Cl₂ (2.00 L) and washed with citric acid (1.00 L*3), brine (500 ml), dried over Na₂SO₄, filtered and concentrated under reduced pressure to get the product. (R)-2-((S)-1-hydroxy-2-(palmitoyloxy)ethyl)-5-oxo-2,5-dihydrofuran-3,4-diyl dinicotinate (150 g, 237 mmol, 28.9% yield, 98.8% purity) was obtained as a light yellow solid, which was confirmed by 1H NMR. HPLC: EW54182-3-P1A1 RT=1.929 min, purity=91.4% (area %); LCMS: EW54182-3-P1A25 RT=0.557 min, MS (ESI) m/z=625.3 [M+1]+; 1H NMR: EW54182-3-P1B12, (400 MHZ, CDCl3) δ 0.78-0.96 (m, 3H), 1.02-1.38 (m, 25H), 1.50-1.70 (m, 2H), 2.20-2.48 (m, 2H), 4.52-4.78 (m, 2H), 5.08-5.24 (m, 1H), 5.82-6.10 (m, 1H), 7.02-7.14 (m, 1H), 7.18-7.26 (m, 1H), 7.60-7.72 (m, 1H), 8.20-8.32 (m, 1H), 8.48 (dd, J=4.80, 1.32 Hz, 1H), 8.52-8.68 (m, 1H), 8.86-9.08 (m, 1H), 9.28-9.52 (m, 1H).

### Compound 15: (R)-2-((R)-1-hydroxy-2-(palmitoyloxy)ethyl)-5-oxo-2,5-dihydrofuran-3,4-diyl dinicotinate

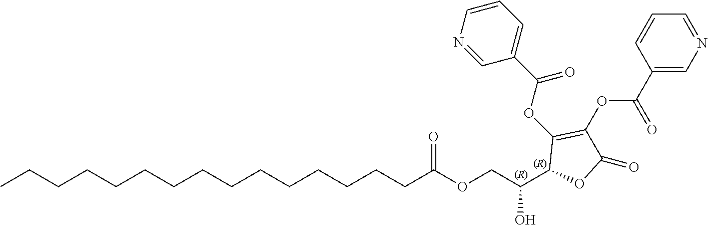

To a solution palmitic anhydride (427 mg, 0.86 mmol, 1.5 equiv.) in DMF (5.5 mL) was added isoascorbic acid 1 (100 mg, 0.58 mmol, 1 equiv.), followed by 4-dimethyl aminopyridine (7 mg, 0.06 mmol, 10 mol %). The reaction mixture was allowed to stir overnight at 25° C. LC-MS (RT=2.191 min) showed isoascorbic acid was consumed completely and desired mass was detected. After lyophilization to remove DMF, the resulting solid was redissolved in 10 mL DCM, washed with water (3×10 mL), dried over sodium sulfate, and concentrated. The resulting residue was purified via C18 reverse-phase chromatography (CH₃CN: H₂O w/0.1% TFA) to obtain (R)-2-((R)-3,4-dihydroxy-5-oxo-2,5-dihydrofuran-2-yl)-2-hydroxyethyl palmitate as a white solid. LCMS: RT=2.191 min, MS (ESI) m/z=415 [M+H]⁺

To a solution of (R)-2-((R)-3,4-dihydroxy-5-oxo-2,5-dihydrofuran-2-yl)-2-hydroxyethyl palmitate (1 g, 2.41 mmol, 1.00 eq) and nicotinoyl chloride hydrochloride 4 (0.95 g, 5.34 mmol, 2.20 eq) in CH₃CN(24 mL) was added N,N-Diisopropylethylamine (2.0 mL, 1.56 g, 12.09 mmol, 5 eq) at 25° C. The mixture was stirred at 25° C. overnight. LC-MS (DG1-125, RT=3.728 min) showed (R)-2-((R)-3,4-dihydroxy-5-oxo-2,5-dihydrofuran-2-yl)-2-hydroxyethyl palmitate was consumed completely and desired mass was detected. The reaction mixture was concentrated under pressure to remove acetonitrile. The remaining solid was redissolved in 10 mL DCM, washed with water (3×10 mL), dried over sodium sulfate, and concentrated. The resulting residue was purified via C18 reverse-phase chromatography (CH₃CN: H₂O w/0.1% TFA) to get (R)-2-((R)-1-hydroxy-2-(palmitoyloxy)ethyl)-5-oxo-2,5-dihydrofuran-3,4-diyl dinicotinate in 87% yield. LCMS: DG1-125-1, product: RT=3.674 min, MS (ESI) m/z=625 [M+H]⁺.

### Compound 16: N6-nicotinoyl-N2-nicotinoylglycyl-L-histidyl-L-lysine

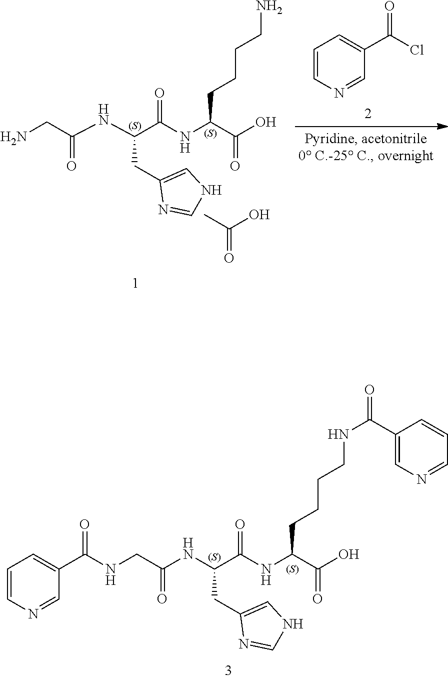

Gly-His-Lys acetate salt (1, 100 mg, 0.25 mmol, 1.00 equiv.) suspended in acetonitrile (6 mL) and cooled to 0° C. and added nicotinoyl chloride 2 (5 equiv) followed by stirring before adding Pyridine (15 equiv.) dropwise at 0° C. The reaction was allowed to warm to 25° C. and stir overnight. LC-MS (RT=0.96 min) indicated the desired compound N6-nicotinoyl-N2-nicotinoylglycyl-L-histidyl-L-lysine 3 with mass (m/z 341.1) was detected. The acetonitrile was removed under pressure, and the resulting residue was purified directly using C₁₈ reverse-phase chromatography to obtain 3 (123 mg, 90%) as a white solid. LCMS: product: RT=9.6 min, MS (ESI) m/z=551.1 [M+H]⁺

### Example 2: Biological Activity of Exemplary Compounds of the Disclosure

### NAD Assay Protocol

Keratinocytes were plated in 12-well tissue culture plates at a density of 200,000 cells per well. The wells contained 0.75 mL DMEM growth medium with 2% HI FBS and cells were incubated overnight at 37° C. at 5% CO₂. The following day, cells were dosed with the desired concentration of each compound and incubated for 3 hours. Following the incubation period, cell monolayers were washed with 200 μL of PBS. Cells were then allowed to lift using 0.25% trypsin for 15 minutes. After lifting, cells were collected into a sterile 1.5 mL centrifuge tube containing 400 μL of DMEM with 2% HI FBS. The cell lysates then centrifuged at 500×g for 5 minutes. Following this, the supernatant was aspirated off of the cell pellet and cells were washed again in 200 μL of PBS and centrifuged at 500×g for 5 minutes. PBS was aspirated off of the cell pellet and 400 μL of cold extraction buffer was added. Each centrifuge tube was then vortexed for 10 seconds. The resulting samples were centrifuged at 13,000×g for 10 minutes at 4° C. to remove insoluble material. After centrifuging, 50 μL of each sample was used to determine in duplicates the total protein concentration by a BCA protein assay kit. The remaining samples were then deproteinized by centrifuging and filtering them through a 10 kDa cut-off spin filter at 13,000xg for 10 minutes at 4° C. Following deproteinization, the samples were loaded into a 96 well plate in duplicates. The amount loaded was dependent on their protein concentration to ensure a uniform protein content for each sample. The final volume in the well was brought up to 50 μL using the cell extraction buffer. 50 μL of each standard was added to the appropriate wells. 98 μL of cycling buffer and 2 μL of NAD cycling enzyme were added to each well and the wells were incubated at room temperature for 5 minutes. 10 μL of developer was added to each well and the plate was incubated at room temperature for one hour. The reaction was stopped by adding 10 μL of stop solution to each well. The absorbance of each well was measured at 450 nm using a plate reader. A standard curve was plotted and normalized NAD values were determined.

TABLE 1
NAD⁺ Increase by Exemplary Compounds

| Compound | NAD+ increase ratio |
|---|---|
| No. | compared to PBS control |
| 1 | 8.19 |
| 2 | 2.53 |
| 3 | 2.77 |
| 4 | 3.04 |
| 5 | 1.51 |
| 6 | 0.99 |
| 7 | 1.5 |
| 8 | 1.5 |
| 9 | 1.67 |
| 10 | 1.57 |
| 11 | 2.7 |
| 12 | 1.16 |
| 13 | 1.67 |
| 14 | 8.33 |
| 15 | 1.84 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound having a structure represented by Formula I or a pharmaceutically acceptable salt thereof: 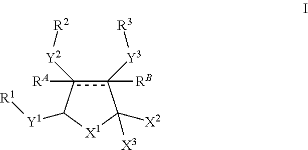
wherein [symbol]
is a single bond or a double bond;
X¹ is O, S, or N(R⁴);
X² and X³ are each independently H, alkyl, or heteroaryl; or X² and X³ combine to form ═O, ═S, or ═N(R⁴);
Y¹ is alkylenyl;
Y² is O, S, or N(R⁵);
Y³ is O, S, or N(R⁶);
R^{A} and R^{B} are each independently absent, H, or alkyl;
R¹ is H, alkyl, alkenyl, alkynyl, cycloalkyl, halo, hydroxy, carboxyl, acyl, acetyl, ester, thioester, alkyl-C(O)—O—, heteroaryl-C(O)—O—, alkoxy, phosphoryl, amino, amido, cyano, azido, or sulfonamido;
R² and R³ are each independently H, alkyl, alkenyl, alkynyl, cycloalkyl, acyl, aryl-C(O)—, heteroaryl-C(O)—, aryl, heteroaryl, aralkyl, heteroaralkyl, or heterocyclyl; and
R⁴, R⁵, and R⁶ are each independently H, alkyl, or aralkyl.

2. The compound of claim 1, wherein [symbol] is a single bond, R^{A} is H, and R^{B} is H.

3. (canceled)

4. (canceled)

5. The compound of claim 1, wherein [symbol] is a double bond; and R^{A} and R^{B} are each absent.

6. The compound of claim 1, wherein X¹ is O.

7. The compound of claim 1, wherein X² is heteroaryl.

8. The compound of claim 1, wherein X³ is H.

9. The compound of claim 1, wherein X² and X³ combine to form ═O.

10. The compound of claim 1, wherein Y¹ is C₁-C₁₀alkylenyl.

11-14. (canceled)

15. The compound of claim 1, wherein R¹ is alkyl-C(O)—O— or heteroaryl-C(O)—O—.

16. (canceled)

17. The compound of claim 1, wherein Y² is O.

18. The compound of claim 1, wherein R² is H.

19. The compound of claim 1, wherein R² is alkyl-C(O)— or heteroaryl-C(O)—O—.

20. (canceled)

21. The compound of claim 1, wherein Y³ is O.

22. The compound of claim 1, wherein R³ is H.

23. The compound of claim 1, wherein R³ is alkyl-C(O)—, heteroaryl-C(O)—, or heterocyclyl.

24-27. (canceled)

28. A compound selected from the group consisting of 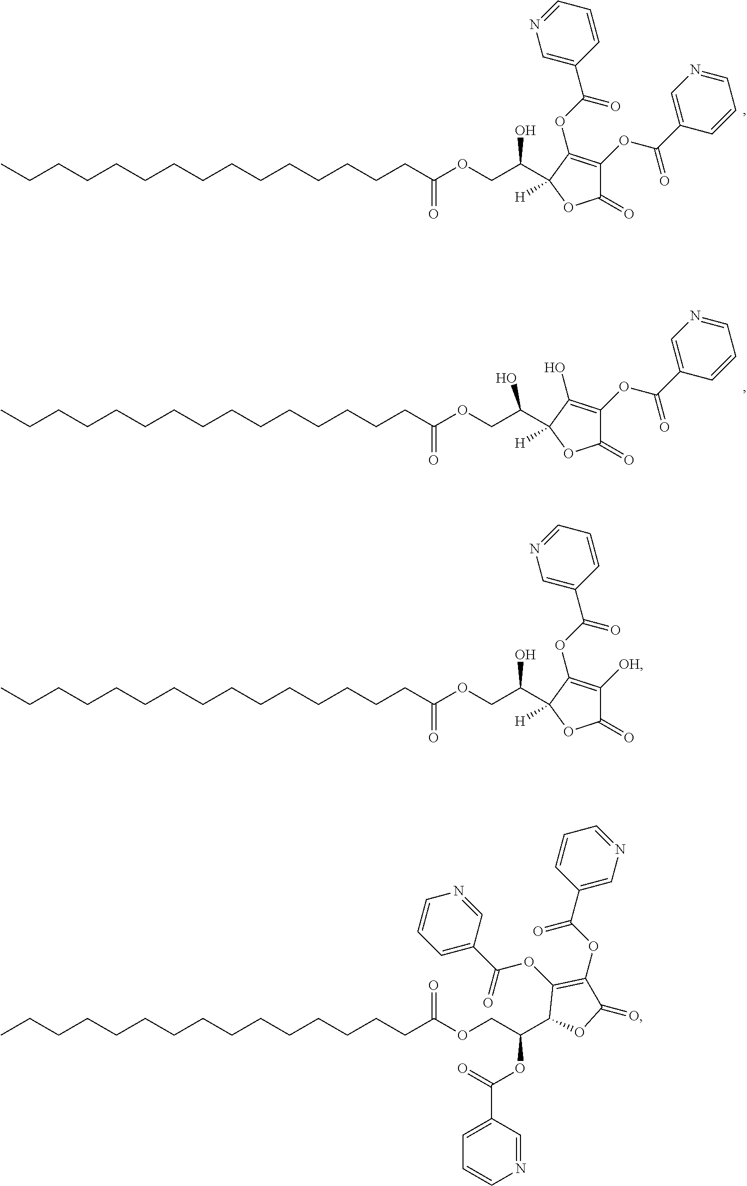 [SMILES: CCCCCCCCCCCCCCCC(=O)OC[C@@H](O)[C@@H]1OC(=O)C(OC(=O)c2cccnc2)=C1OC(=O)c1cccnc1 ; CCCCCCCCCCCCCCCC(=O)OC[C@@H](O)[C@@H]1OC(=O)C(OC(=O)c2cccnc2)=C1O ; CCCCCCCCCCCCCCCC(=O)OC[C@@H](O)[C@@H]1OC(=O)C(O)=C1OC(=O)c1cccnc1 ; CCCCCCCCCCCCCCCC(=O)OC[C@H](OC(=O)c1cccnc1)[C@H]1OC(=O)C(OC(=O)c2cccnc2)=C1OC(=O)c1cccnc1] 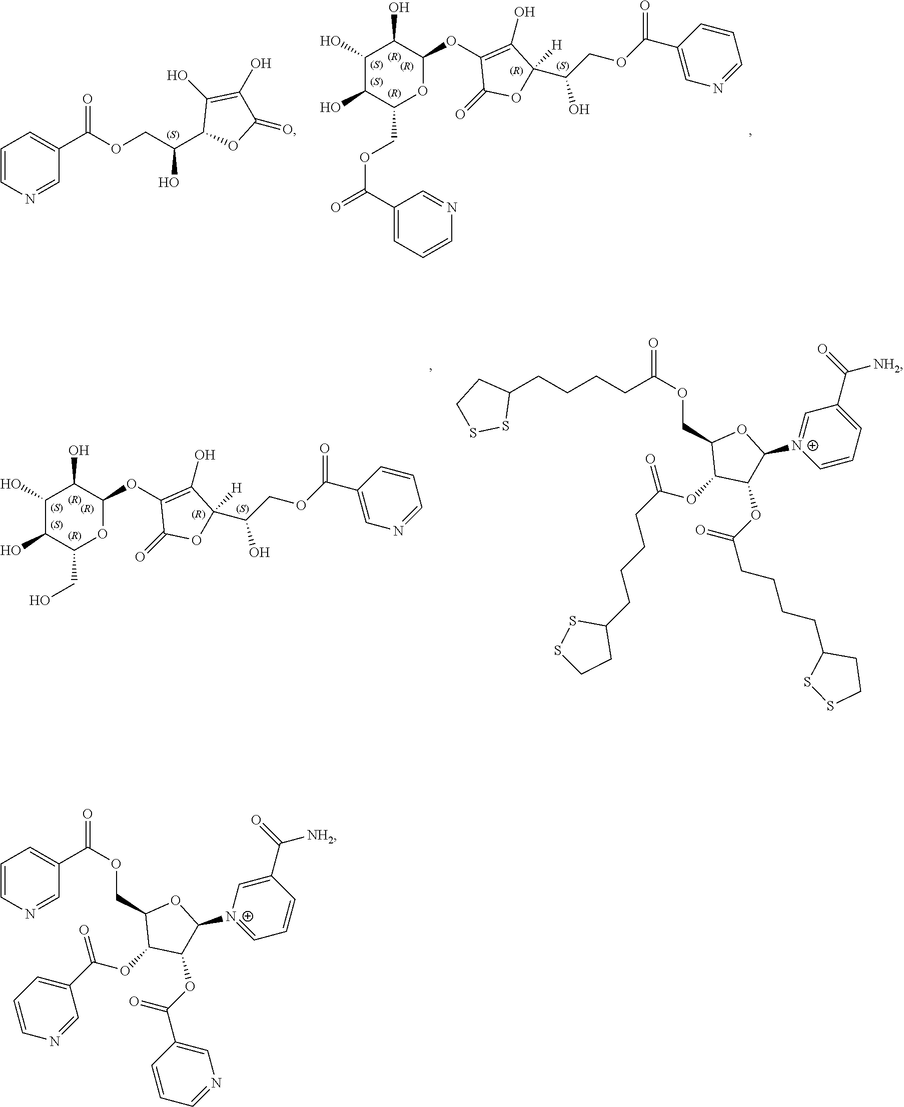 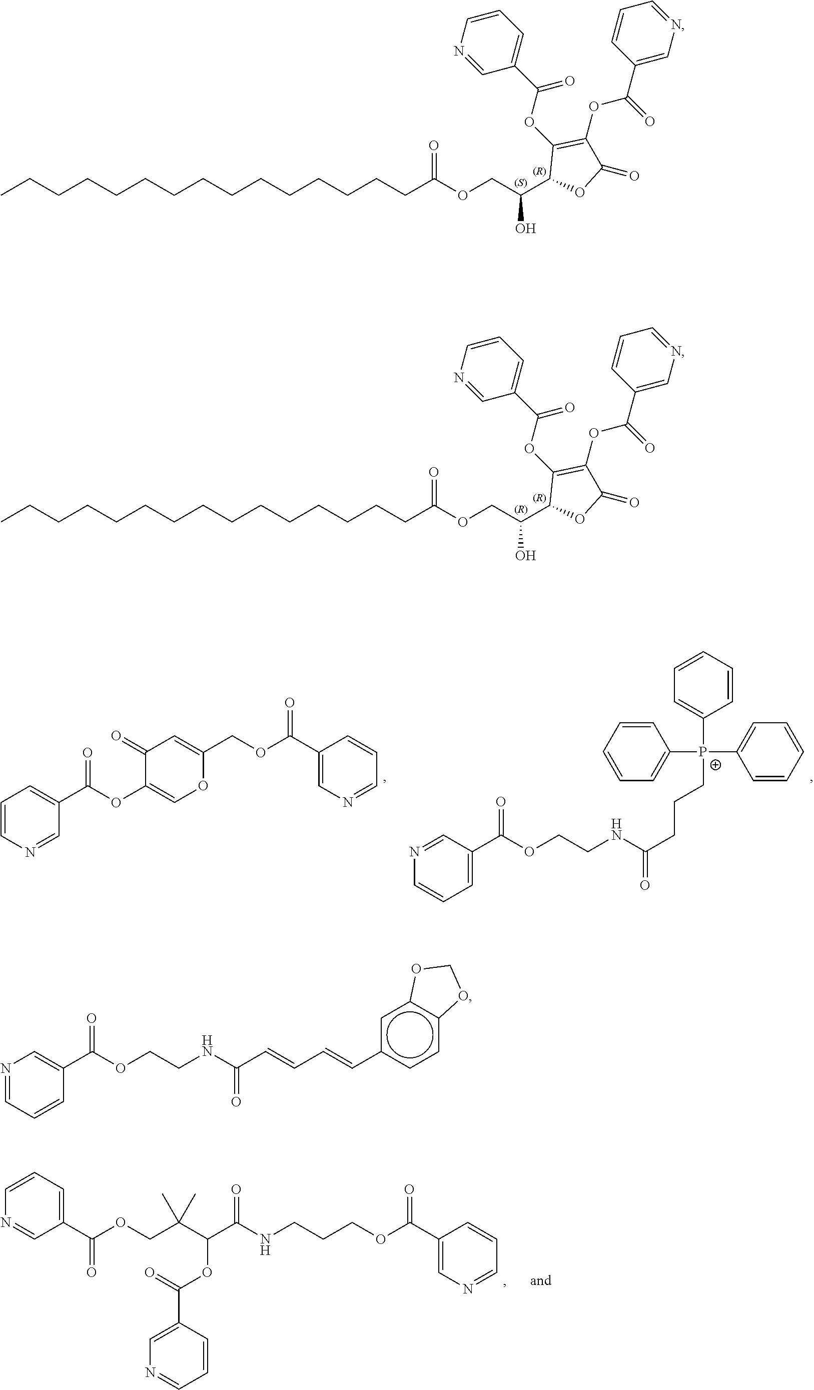 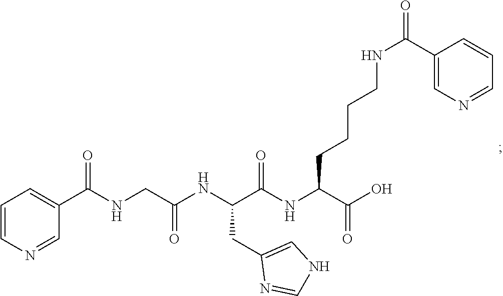 [SMILES: O=C(CNC(=O)c1cccnc1)N[C@@H](Cc1c[nH]cn1)C(=O)N[C@@H](CCCCNC(=O)c1cccnc1)C(=O)O]
or a pharmaceutically acceptable salt thereof.

29-31. (canceled)

32. A pharmaceutical composition comprising the compound of claim 1; and a pharmaceutically acceptable excipient.

33. (canceled)

34. (canceled)

35. A method of increasing intercellular NAD+, comprising administering to a subject in need thereof a compound according to claim 1 in an amount effective to increase the intercellular NAD+ in the subject.

36. (canceled)

37. A method of treating a skin condition in a subject in need thereof, comprising administering to the subject a therapeutically effective amount of a compound of claim 1 or a pharmaceutically acceptable salt thereof.

38-43. (canceled)

44. A method of treating a disease or disorder in a subject in need thereof, comprising administering to the subject a therapeutically effective amount of a compound of claim 1 or a pharmaceutically acceptable salt thereof.

45. (canceled)
